(19) United States
(12) Patent Application Publication
(10) Pub. No.: US 2019/0381211 A1
(43) Pub. Date: Dec. 19, 2019

(54) METHOD OF FORMING A STRUCTURE WITH EXTRACELLULAR MATRIX PROPERTIES IN THE BODY USING TARGETED MICROCAPSULES

(71) Applicants: Mehmet Dogan ASIK, Ankara (TR); Murat BOZKURT, Ankara (TR)

(72) Inventors: Mehmet Dogan ASIK, Ankara (TR); Murat BOZKURT, Ankara (TR)

(21) Appl. No.: 16/323,977

(22) PCT Filed: Aug. 9, 2017
(86) PCT No.: PCT/TR2017/050381

§ 371 Date: Feb. 7, 2019

(30) Foreign Application Priority Data
Aug. 11, 2016 (TR) 2016/11331

(51) Int. Cl. A61L 27/38; A61L 27/58; C12N 5/00

(52) U.S. Cl. CPC A61L 27/3804; A61L 27/3839; C12N 2513/00; C12N 5/0012; A61L 27/58

(57) ABSTRACT

A method of forming a structure with extracellular matrix properties in the body using targeted microcapsules which allow detecting the site of damaged tissues and filling such site with living cells, wherein the method is used in the treatment of damaged tissues and eliminating the need for open surgical intervention.

### TECHNICAL FIELD

The present invention relates to a method of forming a structure with extracellular matrix properties in the body using targeted microcapsules which allow detecting the site of damaged tissues and filling such site with living cells, wherein said method being used in the treatment of damaged tissues and eliminating the need for open surgical intervention.

### PRIOR ART

Tissue engineering is a science field dealing with the formation of organs and tissues in laboratory conditions for the patients needing organ and tissue transplantation. Tissue engineering is an engineering field working on the design and formation of living tissues and organs from living cells and biocompatible/biodegradable polymeric tissue supports. Tissue engineering generally comprises a tissue support that protects and supports cell adhesion and functionality, a rich cell source which is selected in accordance with the target tissue, and growth factors controlling the behavior of such cells.

Bone is a tissue with self-healing and self-regenerating ability. In case of serious damages and segmental fractures, however, such regeneration do not always take place and the regeneration capacity of the bone may not suffice. In such cases in which union will not be possible, filling the bone defects with various materials is of vital importance in order to stimulate bone healing and provide structural and mechanical support in this process.

Articular cartilage is a tissue which is extremely differentiated for fulfilling movement function in humans and has a limited healing property. Its structure is mostly made up of chondrocytes and type II collagens (intercellular network). As the cartilage tissue does not comprise vessels and nerves, it is fed by the so-called perichondrium, dense irregular connective tissue. However, articular cartilage does not comprise perichondrium. Instead, it is supported by synovium, a dynamic tissue located in the inner surface of the joint capsule. Synoviocytes synthesize and secrete the synovial fluid. The chondrocytes within the articular cartilage, unlike many other tissues, are fed by the synovial fluid and predominantly use the anaerobic metabolic pathway. Therefore, the chondrocytes have a limited repairing ability when the cartilage is injured.

Cartilage tissue engineering is defined as a current option for repairing or reforming tissue or organ loss which occurs as a result of a disease or trauma. In cartilage tissue engineering, chondrocytes are taken from the articular cartilage and transferred to a laboratory environment, where they are separated and proliferated in cell culture media. The saturated cartilage-like cells are applied to the injured cartilage site by a second surgical operation. Just as these cells may already be obtained from the cartilage itself, they may be obtained in the form of adult stem cells from bone marrow, periosteum, muscle and fat tissues, and thereafter made into cartilage and used for repairing purposes.

In the state of the art, there exists no method used for the treatment of damaged tissues without requiring open operation. The existing microcapsules cannot be directed to the site of preference with living cells. Again in the state of the art, there is no method of forming a structure which, is delivered to the body without surgical incision or by means of endoscopy, is self-directed to the damaged site with the living cells contained therein, forms a scaffold at the damaged site once it finds such site, and then supports the regeneration of the damaged tissue by polymer chain supplement.

In the method of forming a structure according to the invention, on the other hand, basically the self-renewal means of the body is mimicked. Blood clotting may be given as an example. In case of a vascular injury, thromboplastin enzyme is formed in the blood. This substance is transformed into thrombokinase enzyme by means of a special protein produced by blood platelets. Thrombokinase enzyme transforms the prothrombin from the liver and present in blood plasma, into thrombin. Afterwards, thrombin transforms the fibrinogen, again from the liver, into fibrin, an insoluble fibrous protein. Since fibrin is a fibrous protein, it leads to thrombose by making blood cells precipitate along with it. The method of forming a structure according to the invention uses microcapsule and polymer chain. The site of the damaged tissue is detected by means of the microcapsules injected into the body and cells are transferred to the tissue. A three-dimensional scaffold is formed on the damaged tissue by the microcapsules and polymer chain enclosing these microcapsules, thereby regenerating the tissue. As a result, the damaged tissue is treated with an extracellular matrix-like structure.

### OBJECTS OF THE INVENTION

The object of the present invention is to provide a method of forming a structure with extracellular matrix properties in the body using targeted microcapsules, wherein said method allows tissue regeneration with encapsulated living cells on a damaged tissue at any site of the body.

An object of the invention is to provide a method of forming a structure with extracellular matrix properties in the body using targeted microcapsules, wherein said method allows the regeneration of the damaged tissue without requiring open operation.

Another object of the invention is to provide a method of forming a structure with extracellular matrix properties in the body using targeted microcapsules, wherein said method allows self-renewal of the damaged tissue by forming an extracellular matrix-like structure (scaffold) on the damaged tissue using microcapsule and a polymer.

Another object of the invention is to provide a method of forming a structure with extracellular matrix properties in the body using microcapsules which find the damaged tissue in the body per se and which may be targeted to the damaged tissue.

And another object of the invention is to provide a method of forming a structure which allows self-renewal of the damaged tissue by forming a three-dimensional scaffold with more than one layers in the damaged tissue.

### BRIEF DESCRIPTION OF THE INVENTION

In order to achieve the objects of the present invention, in a method of forming a structure with extracellular matrix properties in the body using targeted microcapsules, which has been defined in the first claim as well as in the other dependent claims, the type of the damaged tissue and the target proteins that permit identifying such tissue are determined. The target protein are present on the damaged tissue and they are tissue-specific. For example, on a damaged cartilage tissue, the antigens that are specific to cartilage tissue are present. The targeting factors that are suitable to said antigens are used in order to identify the damaged tissue, i.e. detect the damaged cartilage tissue. In the present invention, the target proteins are also used for targeting the damaged tissue by the microcapsule.

Said microcapsule comprises living cells. It permits targeting, i.e. finding, the damaged tissue. The microcapsule also adheres to the damaged tissue, thereby forming a scaffold structure on this tissue. The targeting factor on the microcapsule and the target proteins present on the tissue are compatible with one another in terms of structure. Thus, said microcapsule is directed to the damaged tissue and adheres, i.e. attaches, to said damaged tissue only. Once the target proteins are determined, coupling factors are determined by means of the targeting on the microcapsule and the microcapsule is prepared.

The targeting factor enables the microcapsule to detect the damaged tissue. The coupling factor, on the other hand, creates coupling points on the microcapsule for the polymer chain to be linked with the microcapsule. Microcapsule is preferably administered to the body by injection and delivered to the damaged tissue site. After a while following the delivery of the microcapsule to the damaged tissue site, polymer chain is supplemented, and then waiting for a while. The polymer chain not only encloses the microcapsules but also creates a layer to which other microcapsules can adhere. Microcapsule injection and polymer chain supplement continue until the damaged tissue is filled to a sufficient degree. The damaged tissue is completely filled and tissue regeneration is supported thanks to the microcapsule and polymer chain.

### DETAILED DESCRIPTION OF THE INVENTION

The method of forming a structure with extracellular matrix properties in the body using targeted microcapsules, which has been developed for achieving the objects of the present invention, is illustrated in the accompanying drawings, in which:

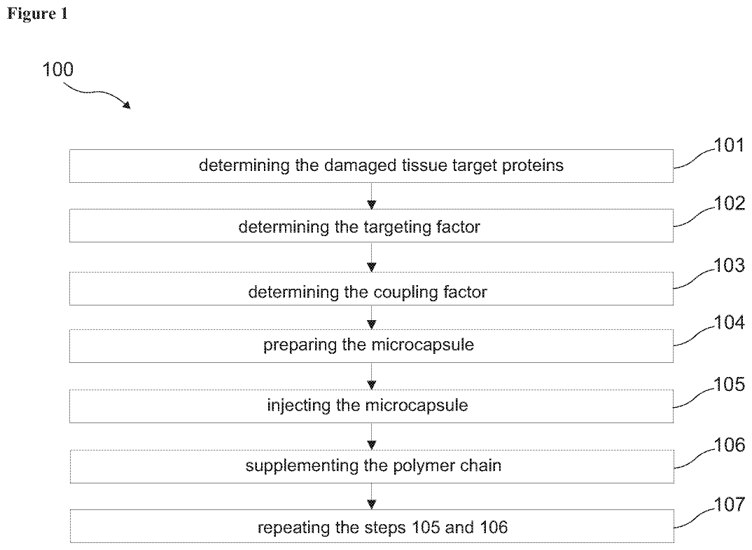

FIG. 1. Schematic view showing the process steps of the method of forming a structure with extracellular matrix properties in the body using targeted microcapsules.

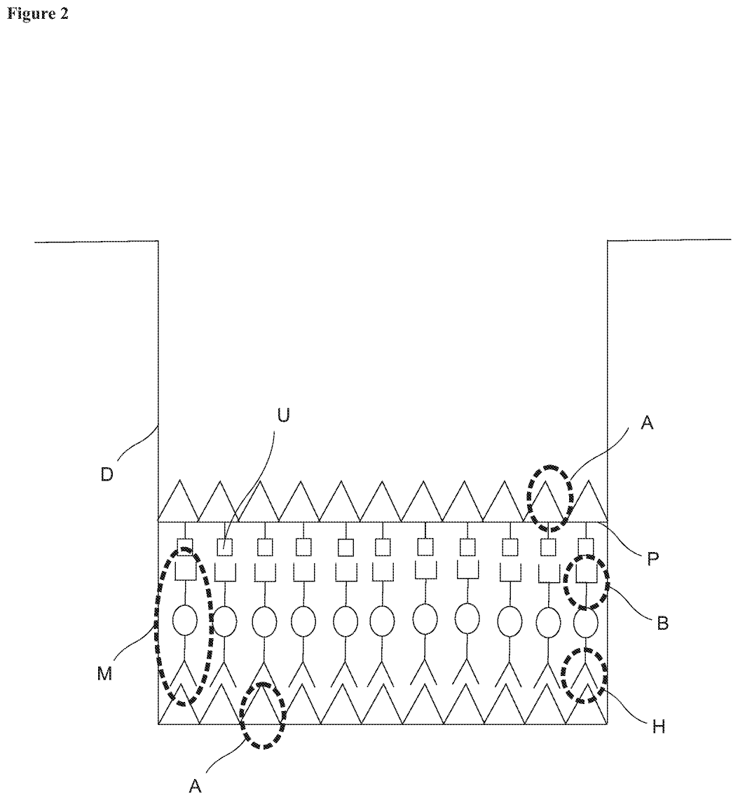

FIG. 2. Schematic view showing the first layer of the scaffold within the damaged tissue.

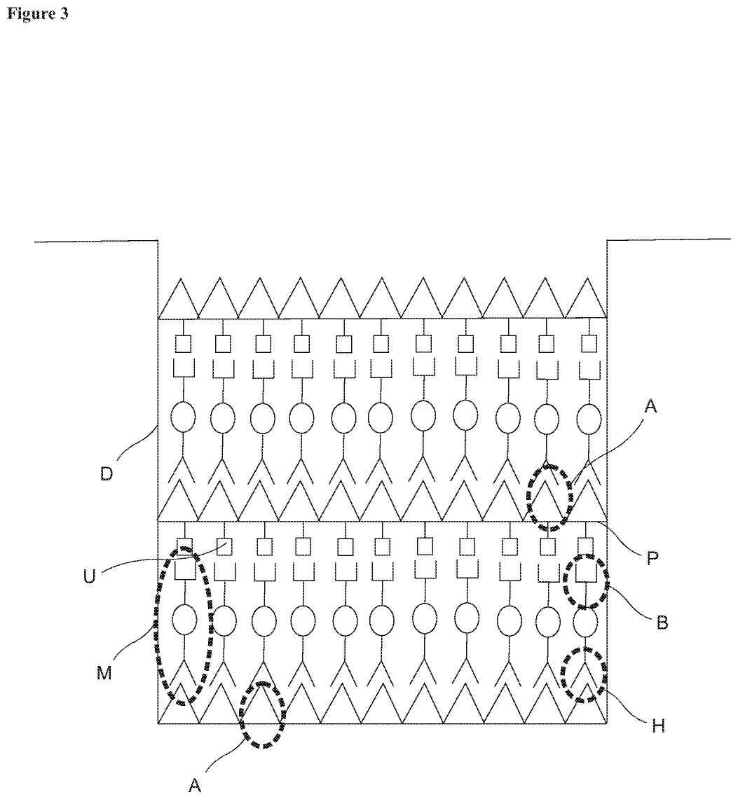

FIG. 3. Schematic view showing the bilayer scaffold.

The parts in the drawings are enumerated individually and the reference numerals corresponding thereto are given below.
100. Method of forming a structure
   D. Damaged tissue
   M. Microcapsule
   A. Target protein
   H. Targeting factor
   B. Coupling factor
   U. Coupling end
   P. Polymer chain

The method (100) of forming a structure with extracellular matrix properties in the body using targeted microcapsules (M) which allow detecting the site of damaged tissues (D) and filling such site with living cells, without surgical incision or by means of endoscopy, comprises the process steps of:
determining the tissue-specific target proteins (A) present on the damaged tissue (D) to be filled (101),
determining the targeting factor (H) which is compatible with the target proteins (A) present on the damaged tissue (D) and enables the microcapsule (M) to be directed to damaged tissue (D) (102),
determining the coupling factors (B) present on the microcapsule (M) and allowing the polymer chain (P) to be attached to the microcapsule (M) (103),
preparing a microcapsule comprising living cells, targeting factor (H), and coupling factor (B) (104),
injecting the microcapsule (M) to the damaged tissue (D) (105),
supplementing the polymer chain (P) comprising target proteins (A) and coupling factors (B), said polymer chain adhering to the microcapsule (M) and forming the basis for the multiple-layer structure (106), and
repeating the steps 105 and 106 until the damaged tissue (D) site is filled (107).

The method (100) of filling damaged tissues (D) with targeted microcapsules (M) according to the invention allows access to the damaged site within the body without open surgical intervention. The regeneration of the damaged tissue is provided by the method (100) of filling damaged tissues (D). To that end, the microcapsules (M) carrying living cells are delivered to the damaged site and a scaffold structure with extracellular matrix properties is formed at that site. The targeted microcapsules (M) are preferably injected to the damaged site. The microcapsules (M) carrying living cells reach the damaged site and adhere to the damaged tissue (D) in order to form the scaffold system required for regeneration. In an embodiment of the invention, the method according to the invention is used for filling a damaged cartilage tissue. This embodiment is carried out at a damaged site on the cartilage tissue. The microcapsules (M) are infected to the damaged site. The microcapsules (M) delivered to the body find the damaged site and adhere thereto.

The method according to the invention may be used for the regeneration of damaged tissues (D) of different types.

In the method (100) of forming a structure, first the efforts on how to detect/identify the damaged tissue (D) are made. For example, a damaged site on the cartilage tissue is identified by target proteins specific to that region. To that end, the target proteins (A) belonging to the tissue which will be regenerated/filled are determined (101). The target proteins (A) are preferably present on the exposed surface of the damaged tissue (D). The site at which the target proteins (A) are present can be interpreted as a damaged one. In case of any damage to a tissue, the target proteins (A) present on that tissue become exposed on the tissue surface. In the method according to the invention, the target proteins (A) are used for detecting the damaged tissue and making the microcapsule (M) adhere to said damaged tissue (D). The target proteins (A) differ in terms of their properties according to each tissue type. In other words, the target protein (A) of each tissue is specific to that tissue.

Once the target proteins (A) of the damaged tissue (D) are determined (101), the targeting factor (H) which will allow regeneration on the damaged tissue (D) is determined (102). The targeting factors (H) are present on the microcapsule (M). The targeting factors (H) are selected according to the target proteins (A) present on the damaged tissue (D). During the selection of the targeting factors (H), such factors as DNA sequence, the antibody compatible with the target proteins (A) present on the damaged tissue (D), etc. are selected. In a preferred embodiment of the invention, an antibody is used as the targeting factor (H). In this case, an antibody specific to cartilage is selected as the targeting factor (H) compatible with the target protein (A) present on the cartilage tissue. The targeting factor (H) ensures that the microcapsule (M) is directly oriented to the damaged tissue (D) and adheres to the target proteins (A) present on the damaged tissue (D). The targeting factor (H) is present on the microcapsule (M).

When the targeted microcapsule (M) adheres to the damaged tissue (D), coupling points on the microcapsule (M) are required in order that the polymer chain (P) can be linked to the microcapsule (M) and the site at which said microcapsule (M) is present. Coupling factors (B) are used so that the polymer chain (P) can be attached to the microcapsules (M) present on the damaged tissue (D). The coupling factors (B) allow the polymer chain (P) to be attached to the microcapsule (M). In the method according to the invention, the coupling factors (B) are present on the microcapsule (M). Thus, coupling points are also obtained in the upper portion of the microcapsules (M) and that region is enclosed with the polymer chain (P). The coupling factor (B) is determined in order for the targeted microcapsule (M) to form coupling points on the damaged tissue (D) (103). Once the targeted microcapsule (M) adheres to the target proteins (A) present on the damaged site, the polymer chain (P) is injected to form a scaffold structure. The fact that the polymer chain (P) adheres to or encloses the microcapsules (M) is ensured by the coupling factors (B). The coupling factor (B) may be an antibody, DNA sequence, or aptamer. In an embodiment of the invention, the coupling factor (B) is a rhodopsin-specific antibody, a selected protein.

Once the targeting factor (H) and the coupling factor (B) are determined (102 and 103), the microcapsule (M) is prepared (104). During microcapsule (M) preparation (104), first the living cells are encapsulated with a polymer, preferably alginate, and the primary layer with side groups thereon is obtained. In this step, preferably CaCl₂) dissolved in water and a low molecular weight sodium alginate polymer again dissolved in water are cross linked. Preferably the chondrocytes proliferated in the cell culture are added into the alginate solution and instilled into the CaCl₂) solution. The cell is thus coated with the primary layer. Following the cell encapsulation, the polymer, preferably chitosan, is functionalized and the bridging factor, preferably avidin, is bound to the functionalized chitosan. In this step, the chitosan is functionalized and avidinated. First of all, low molecular weight chitosan polymer is dissolved in acetic acid solution. And then, the ambient pH value is increased to the physiological pH range. Once the pH value is increased, chitosan is first treated with functionalizing agent, and then with avidin molecules. Avidin-bound chitosan polymer chains are washed with acetic acid solution at least once. After the functionalization of the secondary polymer, preferably chitosan, and the binding of the bridge factor, preferably avidin, the encapsulated cells are coated with a functionalized polymer in order to form the second layer thereof. Encapsulated cells are washed with the preferred isotonic solution. In this embodiment of the invention, the cells are preferably washed with PBS (Phosphate Buffered Saline) buffer solution and transferred to the PBS solution. After washing, the avidin-bound chitosan polymers are brought to pH 4.5 and introduced into the PBS solution (pH 7.4) in which encapsulated cells are present. After coating the encapsulated cells with the functionalized polymer to form the second layer, the second layer and the microcapsule (M) are obtained by binding, on the functionalized polymer, preferably the biotinylated targeting factor (H) and the growth/differentiation factor. The solution comprising therein biotinylated antibodies specific to the damaged cartilage, e.g. glycoprotein, 80 anti-GPNMB, anti-CD90/THY1, anti-GPCR, anti-S100A9, anti-CXCR4, and anti-periostin, is instilled into the solution in which the encapsulated cells are present. Once the instillation is performed, it is kept for 0-24 hours at 30-45° C. The avidinated chitosan is also treated with biotinylated rhodopsin for 0-24 hours in a different environment.

The microcapsules (M) the outer surface of which is coated with antibodies are injected to the damaged tissue (D) site (105). After the injection, they are kept for a time period of 0-24 hours. This is the incubation period of the microcapsule (M). During this period, the microcapsule (M) detects the damaged tissue thanks to the targeting factors (H) and is located at a suitable position on the tissue, and then being stabilized. Again during this period, the microcapsule (M) binds to the target proteins (A) on the damaged tissue (D) by means of the targeting factors and forms coupling points on the damaged tissue (D). As a result of injection, the microcapsules (M) are coupled to the target proteins (A) present on the damaged tissue (D) by means of the targeting factors (H). The binding factors (B) present on the microcapsules (M) are waited in an exposed manner.

Once the waiting time after microcapsule (M) injection (105) to the damaged site is over, polymer chain (P) supplement (106) is performed. Polymer chain (P) supplement (105) is performed under predetermined pH conditions. The supplemented polymer chain (P) may be different from or the same as the polymer present on the microcapsule (M). In the preferred embodiment of the invention, the supplemented polymer chain (P) is the same as the polymer present on the microcapsule (M). In an embodiment of the invention, the polymer chain (P) is chitosan. The polymer chain (P) is provided thereon with target proteins (A) and coupling ends (U). The coupling ends (U) present on the polymer chain (P) are compatible with or the same as the coupling factors (B) present on the microcapsule (M). The coupling ends (U) present on the polymer chain (P) enable the polymer chain (P) to adhere to the coupling factors on the microcapsule (M). There exist more than one coupling ends (U) on the polymer chain (P). Thanks to these coupling ends (U), each microcapsule (M) is individually bound to a polymer chain (P). Polymer chain (P) also comprises target proteins (A). Such target proteins (A) enable the microcapsule (M) to identify or adhere to the polymer chain (P). Thus, thanks to the polymer chain (P), not only a binding to the microcapsules (M) which adhere to the damaged tissue (D) is performed but also a basis comprising target protein (A), to which new microcapsules (M) may be linked, is formed by enclosing the microcapsules (M).

The steps 105 and 106 are repeated until the next layer on the damaged tissue (D) is formed (107). As the steps 105 and 106 are repeated, a scaffold structure is formed. During the first microcapsule (M) and polymer chain (P) injection, the microcapsules (M) adhere to the target proteins present on the damaged tissue (D) and the polymer chain (P) encloses the microcapsules (M) adhering to the target proteins (A). In the following steps, the injected microcapsules (105) adhere to the previously added polymer chains (P). During each repetition of the steps 105 and 106 (107), a new layer is formed on the damaged tissue (D) and thus, a three-dimensional scaffold system is created on the damaged tissue (D).

In an alternative embodiment of the invention, the microcapsules (M) in which no living cells are present are delivered over the damaged tissue (D). After the microcapsules (M) in which no living cells are present are located on the damaged tissue (D), the polymer chain (P) is injected. Upon attachment of the polymer chain (P) to the microcapsules (M) in which no living cells are present, the microcapsules (M) comprising living cells are injected. The microcapsules (M) comprising living cells are located on the polymer chain (P). This process is repeated as many times as required; thus, an extracellular matrix-like structure is formed on the damaged tissue (D).

In an alternative embodiment of the invention, nanoparticles are delivered to the damaged tissue (D) instead of microcapsules and the scaffold structure is formed as such.

In the method of forming a structure (100), which is the subject of the present invention, a damaged tissue due to any reason is filled with microcapsules (M) comprising living cells and polymer chains (P) thereby forming a scaffold. To that end, first the target proteins (A) present on the damaged tissue (D) are determined (101). After the determination of the target proteins (A) specific to the damaged tissue (D) (101), the targeting factors (H) that are compatible with the target proteins (A) and allow directing to the target proteins (A) are determined (102). The targeting factors (H) are present on the microcapsules (M). Present on the microcapsules (M), in addition to the targeting factors (H), are coupling factors (B). The coupling factors (B) form coupling points on the microcapsules (M) and allow the polymer chain (P) to be attached to the microcapsules (M). Therefore, the next step is the determination of the coupling factors (B) (103). After the target proteins (A), targeting factors (H), and coupling factors (B) are determined (101, 102, and 103), the microcapsules (M) that will permit the living cells to reach the damaged tissue (D) are prepared (104). Once prepared, the microcapsules (M) are delivered to the body by means of various methods, preferably by injection (105). The microcapsules (M) delivered into the body are directed towards the target proteins (A) present on the damaged tissue (D), and then adhere to said damaged tissue (D). Once the microcapsules (M) are stabilized on the damaged tissue (D), the polymer chain (P) is delivered to the body (106). The coupling ends (U) compatible with the coupling factors (B) on the microcapsule (M) are present on the polymer chain (P). Hence, the polymer chain (P) encloses the previously delivered microcapsules (M). Polymer chain (P) also comprises target proteins (A). In the next microcapsule (M) injection, the target proteins (A) present on the polymer chain (P) form the points to which the microcapsules (M) adhere. Microcapsule (M) injection (104) and polymer chain (P) supplement (106) as many times as preferred may be performed on the damaged tissue (D) (107). In this way, a three-dimensional scaffold system is formed on the damaged tissue (D), thereby regenerating the tissue.

1. A method (100) of forming a structure which allows detecting the site of damaged tissues (D) and filling such site with living cells, without surgical incision or by means of endoscopy, comprising the process steps of:
determining the tissue-specific target proteins (A) which are present on the exposed damaged tissue (D) to be filled (101), and which enable to detect that the said site is damaged, and
determining the targeting factor (H) which is compatible with the target proteins (A) present on the damaged tissue (D) and enables the microcapsule (M) to be directed to damaged tissue (D) (102),
characterized by the process steps of
determining the coupling factors (B) present on the microcapsule (M) and allowing the polymer chain (P) to be attached to the microcapsule (M) (103),
preparing a microcapsule (M) comprising living cells, targeting factor (H), and coupling factor (B) (104), wherein
the living cells are encapsulated with a polymer, preferably alginate, and the primary layer with side groups thereon is obtained,
the polymer, preferably chitosan, is functionalized and the bridge factor, preferably avidin, is bound to the functionalized chitosan and dissolved in the acetic acid solution,
the encapsulated cells are coated with the functionalized polymer in order to obtain the second layer,
the second layer and the microcapsule (M) are obtained by binding, on the functionalized polymer, preferably the biotinylated targeting factor (H) and the tissue accumulation factor;
injecting the microcapsule (M) to the damaged tissue (D) (105), wherein
the microcapsule (M) is bound to the damaged tissue (D) by means of the targeting factor (H) and forms the points on the damaged tissue (D) to which the polymer chain (P) can be bound, with the coupling factors (B),
the microcapsule (M) adheres to the damaged tissue (D) and forms, within the damaged tissue, a scaffold structure comprising living cells,
supplementing the polymer chain (P) comprising target proteins (A) and coupling ends (U), said polymer chain adhering to the microcapsule (M) and forming the basis for the multiple-layer structure (106), wherein
the polymer chain (P) encloses the microcapsules (M) such that a basis comprising target protein (A), to which new microcapsules (M) can be bound, is formed,
a scaffold structure is formed within the damaged tissue (D), with the microcapsule (M) binding to the target proteins (A) and the polymer chain (P) binding to the microcapsule (M).

2. (canceled)

3. The method (100) of forming a structure as in claim 1, characterized in that during the process step of preparing the microcapsule (M) (104), the CaCl₂) dissolved in water and a low molecular weight sodium alginate polymer dissolved in water are cross linked.

4. The method (100) of forming a structure as in claim 3, characterized in that during the process step of preparing the microcapsule (M) (104), preferably the chondrocytes proliferated in the cell culture are added into the alginate solution, and then the chondrocytes added into the alginate solution are instilled into the CaCl2 solution.

5. (canceled)

6. The method (100) of forming a structure as in claim 3, characterized in that during the process step of preparing the microcapsule (M) (104), the pH value is brought to physiological pH range and treatment with bridge factor molecules, preferably with avidin, is performed.

7. The method (100) of forming a structure as in claim 6, characterized in that during the process step of preparing the microcapsule (M) (104), the polymer chain (P), preferably chitosan, is washed with acetic acid at least once.

8. (canceled)

9. The method (100) of forming a structure as in claim 7, characterized in that during the process step of preparing the microcapsule (M) (104), the encapsulated cells are washed with an isotonic solution, preferably phosphate buffered saline (PBS) buffer solution, and then transferred to the PBS solution.

10. The method (100) of forming a structure as in claim 9, characterized in that during the process step of preparing the microcapsule (M) (104), the pH value of the avi din-bound chitosan polymers is brought to 4.5 and it is introduced into the PBS solution in which encapsulated cells are present.

11. (canceled)

12. The method (100) of forming a structure as in claim 10, characterized in that during the process step of preparing the microcapsule (M) (104), the solution comprising therein antibodies of glycoprotein, 80 anti-GP MB, anti-CD90/THY1, anti-GPCR, anti-S100A9, anti-CXCR4, and anti-periostin, is instilled into the solution in which the microcapsules (M) are present.

13. The method (100) of forming a structure as in claim 12, characterized in that during the process step of preparing the microcapsule (M) (104), subsequent to the completion of the instillation process, it is waited for 0-24 hours at 30-45° C. and then the avidinated chitosan is treated with biotinylated rhodopsin for 0-24 hours.

14. (canceled)

15. The method (100) of forming a structure as in claim 1, characterized in that during the process step of injecting the microcapsule (M) (105), the microcapsules (M) the outer surface of which is coated with antibodies are delivered to the damaged site and subsequent to delivery, the microcapsule (M) is waited during the incubation period so that it can be located in a suitable place and stabilized therein.

16. (canceled)

17. (canceled)

18. The method (100) of forming a structure as in claim 1, characterized in that during the process step of supplementing the polymer chain (P) (106), once the incubation period is over, the polymer chain (P) is delivered onto the microcapsules (M) reaching the damaged.

19. (canceled)

20. The method (100) of forming a structure as in claim 1, characterized in that during the process step of repeating the steps 105 and 106 (107), said steps 105 and 106 are repeated until the preferred amount of tissue is formed on the damaged tissue (D), i.e. until the damaged tissue is completely filled, and a scaffold structure is formed within the damaged tissue (D).

21. The method (100) of forming a structure as in claim 20, characterized in that during the process step of repeating the steps 105 and 106 (107), the multi-layer scaffold structure is formed by further supplementing, onto the scaffold structure within the damaged tissue (D), the microcapsule (M) binding to the target proteins (A) and the polymer chain (P) binding to the microcapsule (M).

22. The method (100) of forming a structure as in claim 1, characterized in that during the process step of determining the targeting factor (H) (102), when selecting the targeting factors (H); DNA sequence, aptamer factors, and the antibody compatible with the target proteins (A) present on the damaged tissue (D) are selected.
